(19) United States
(12) Patent Application Publication
(10) Pub. No.: US 2010/0056820 A1
(43) Pub. Date: Mar. 4, 2010

(54) Method for the production of substituted 2-aryl malonic acid esters

(75) Inventors: Volker Maywald, Ludwigshafen (DE); Christian Ott, Speyer (DE); Bernd Wolf, Fussgönheim (DE); Manfred Ehresmann, Maxdorf (DE); Michael Rack, Eppelheim (DE); Michael Keil, Freinsheim (DE); Sebastian Peer Smidt, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/522,211

(22) PCT Filed: Jan. 21, 2008
(86) PCT No.: PCT/EP08/50651

§ 371 Date: Jul. 6, 2009

(30) Foreign Application Priority Data
Jan. 22, 2007 (EP) 07100926.0

(51) Int. Cl. C07C 69/34

(52) U.S. Cl. 560/96

(57) ABSTRACT

The present invention relates to a process for preparing substituted 2-arylmalonic esters of the general formula I

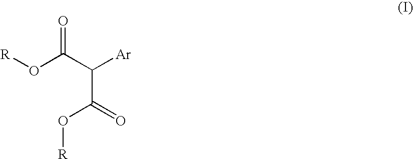

in which R is C₁-C₆-alkyl or C₁-C₄-alkoxy-C₁-C₄-alkyl; Ar is phenyl or a heteroaromatic 5- or 6-membered ring; where each carbon atom present in the radicals mentioned above optionally carries a substituent R^{A}; R^{A} is F, Cl, CN, NO₂, C₁-C₄-alkyl, C₁-C₄-haloalkyl, C₁-C₄-alkoxy, C₁-C₄-haloalkoxy, etc., or two adjacent substituents R^{A} together with the carbon atoms to which they are attached form a ring; and where a malonic ester is reacted with a base and an aryl bromide in the presence of a copper salt, which comprises employing from 0.1 to 0.65 molar equivalents of the base per molar equivalent of the malonic ester.

The present invention relates to a process for preparing substituted 2-arylmalonic esters which comprises reacting a malonic ester with a base and an aryl bromide in the presence of a copper salt.

Substituted 2-arylmalonic esters are useful intermediates in the preparation of numerous organic compounds such as, for example, agrochemicals or pharmaceutics and in particular in the preparation of fungicidal triazolopyrimidines as described, for example, in EP 0 550 113, EP 0 782 997, EP 0 770 615, EP 0 975 634 or WO 98/46607.

From the prior art, the preparation of substituted 2-arylmalonic esters is known in principle. Thus, DE 199 38 736, for example, describes a process for preparing bis(trifluoromethyl)phenylacetic acids and alkyl esters thereof by decarboxylation of dialkyl bis(trifluoromethyl)phenylmalonate intermediates. For the preparation of dialkyl malonates, DE 199 38 736 teaches the reaction of a corresponding phenyl bromide or phenyl iodide with a dialkyl malonate in the presence of a deprotonating agent, a copper salt and a solvent.

EP 1 002 788 and U.S. Pat. No. 6,156,925 describe a process for preparing 2-phenylmalonic esters which comprises reacting a molar equivalent of a phenyl bromide with 2 to 4 molar equivalents of a dialkyl malonate in an inert solvent in the presence of from 2 to 3.8 molar equivalents of a base, especially NaH, and a copper salt. The base is employed in approximately equimolar amounts, based on the malonic ester.

Owing to the solvents and reagents used, work-up of the reaction mixtures obtained according to the prior art is expensive and complex.

Accordingly, it is an object of the present invention to provide a process which, by virtue of reduced expense during work-up, is suitable especially for an industrial production of substituted 2-phenylmalonic esters, affording these compounds in high yield and purity.

Surprisingly, it has been found that this object is achieved by using a significant excess of malonic ester, based on the base employed.

Accordingly, the present invention provides a process for preparing substituted 2-arylmalonic esters of the general formula I

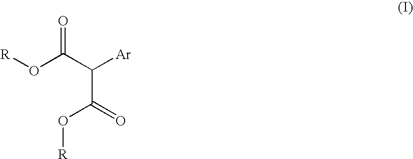

in which
R is C₁-C₆-alkyl or C₁-C₄-alkoxy-C₁-C₄-alkyl; and
Ar is phenyl or a heteroaromatic 5- or 6-membered ring comprising, as ring members, 1 or 2 heteroatoms selected from the group consisting of N, S and O, where each carbon atom present in the radicals mentioned above optionally carries a substituent R^{A}, where
   R^{A} independently of one another is fluorine, chlorine, cyano, nitro, C₁-C₄-alkyl, C₁-C₄-haloalkyl, C₁-C₄-alkoxy, C₁-C₄-haloalkoxy, C₁-C₆-alkoxycarbonyl, C₁-C₆-alkylaminocarbonyl or di-(C₁-C₆-alkyl)aminocarbonyl; or
      two adjacent substituents R^{A} together with the carbon atoms to which they are attached form an aromatic or partially saturated optionally substituted 5- to 7-membered ring; and
         where a malonic ester of the general formula II

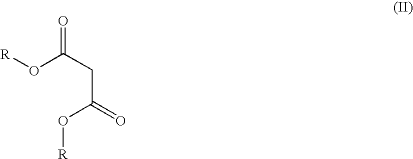

in which R is as defined above is reacted with a base and an aryl bromide of the formula III

Ar-Br (III)

in which Ar is as defined above in the presence of a copper salt, which comprises employing from 0.1 to 0.65 molar equivalents of the base per molar equivalent of the malonic ester of the formula II.

Used in the definition of the substituents for organic groups are collective terms which represent the individual members of these groups of organic moieties. In the particular case, the prefix Cₓ-C_{y} denotes the number of possible carbon atoms.

The term “C₁-C₆-alkyl”, as used herein and in the terms C₁-C₆-alkylaminocarbonyl and di(C₁-C₆-alkyl)aminocarbonyl, denotes a saturated straight-chain or branched hydrocarbon group comprising 1 to 6 carbon atoms, especially 1 to 4 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethyl-propyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and their isomers. C₁-C₄-Alkyl includes, for example, methyl, ethyl, propyl, 1-methylethyl, butyl, 1-methylpropyl, 2-methylpropyl or 1,1-dimethylethyl.

The term “C₁-C₄-haloalkyl”, as used herein and in the haloalkyl moieties of C₁-C₄-haloalkoxy, describes straight-chain or branched alkyl groups having 1 to 4 carbon atoms, where some or all of the hydrogen atoms of these groups are replaced by halogen atoms, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, etc.

The term “C₁-C₄-alkoxy”, as used herein and in the alkoxy moieties of C₁-C₄-alkoxy-C₁-C₄-alkyl and C₁-C₄-alkoxycarbonyl, describes straight-chain or branched saturated alkyl groups comprising 1 to 4 carbon atoms, which groups are attached via an oxygen atom. Examples include C₁-C₄-alkoxy, such as, for example, methoxy, ethoxy, OCH₂—C₂H₅, OCH(CH₃)₂, n-butoxy, OCH(CH₃)—C₂H₅, OCH₂—CH(CH₃)₂, OC(CH₃)₃.

The term “C₁-C₄-haloalkoxy”, as used herein, describes C₁-C₄-alkoxy groups as described above where some or all of the hydrogen atoms of these groups are replaced by halogen atoms, such as chloromethoxy, dichloromethoxy, trichloro-methoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-tri-chloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy or nonafluorobutoxy.

The term “C₁-C₄-alkoxy-C₁-C₄-alkyl” describes an alkyl group having 1 to four carbon atoms in which one hydrogen atom is replaced by an alkoxy group having 1 to four carbon atoms. Examples include methoxymethyl, ethoxymethyl, —CH₂OCH₂—C₂H₅, —CH₂—OCH(CH₃)₂, n-butoxymethyl, —CH₂—OCH(CH₃)—C₂H₅, —CH₂—OCH₂—CH(CH₃)₂, —CH₂—OC(CH₃), methoxyethyl, ethoxyethyl, —(CH₂)₂OCH₂—C₂H₅, —(CH₂)₂OCH(CH₃)₂, n-butoxyethyl, —(CH₂)₂OCH(CH₃)—C₂H₅, —(CH₂)₂OCH₂—CH(CH₃)₂ or —(CH₂)₂—OC(CH₃), etc.

The term “heteroaromatic 5- or 6-membered ring” describes a cyclic group comprising, as ring member, at least one heteroatom selected from the group consisting of N, O and S and at least two conjugated C═C or C═N double bonds. Examples of these are furanyl, pyrrolyl, imidazolyl, pyrazolyl, thiazolyl, isothiazolyl, isoazolyl, pyridinyl, pyrazinyl, pyrimidinyl, pyridazinyl, etc.

The substances employed in the process according to the invention are advantageously used in sufficiently high purity. The malonic ester of the formula II is preferably essentially anhydrous, i.e. the water content of the malonic ester is preferably below 500 ppm. This applies analogously to the base employed, i.e. the latter should preferably have a water content or a content of hydrolyzed base of less than 1.5% by weight. The copper salt used has preferably a purity of at least 99% by weight; the content of Cu²⁺ impurities is preferably less than 0.5%.

In a special embodiment of the process according to the invention, the malonic ester of the general formula II is reacted with a base and the reaction product obtained is reacted in the presence of the copper salt with the aryl bromide of the general formula III.

In a preferred embodiment of the process according to the invention, the reaction is carried out essentially without addition of an inert solvent. In particular, in the process according to the invention, the reaction mixture comprises less than 20% by weight, preferably less than 10% by weight and particularly preferably less than 2% by weight of an inert solvent. In the process according to the invention, the reaction is especially carried out neat, i.e. without addition of an inert solvent.

Surprisingly, it has been found that under the reaction conditions described above there is no increase in unwanted condensation reactions of the malonic ester used, or they do not occur to an extent which is inconvenient.

In the context of the present invention, the term “inert solvent” refers to organic compounds or mixtures thereof added to a reaction without these compounds being involved in the reaction in any significant manner or without these compounds being chemically modified in the reaction. In the case of the process according to the invention, such inert solvents are, for example, aliphatic or aromatic hydrocarbons, such as n-hexane, cyclohexane, toluene or xylenes, halogenated hydrocarbons, such as dichloromethane or chloroform, aromatic chlorinated hydrocarbons, such as chlorobenzene, ethers, such as diisopropyl ether, tert-butyl methyl ether, tetrahydro-furan or dioxane, or amides, such as N-methylformamide.

In the process according to the invention the base is employed in a substoichiometric amount, based on the malonic ester of the formula II. The base is preferably employed in an amount of from 0.1 to 0.6 molar equivalents, in particular from 0.3 to 0.58 molar equivalents and especially from 0.4 to 0.55 molar equivalents, based on 1 mole of the malonic ester of the formula II.

In the process according to the invention, if the malonic ester used is diethyl malonate, the base is particularly preferably employed in an amount of from 0.3 to 0.55 molar equivalents, based on 1 mole of diethyl malonate.

In the process according to the invention, the malonic ester of the formula II is preferably employed in an amount of from 1.5 to 20 molar equivalents, in particular from 1.5 to 10 molar equivalents and especially from 1.5 to 5 molar equivalents, based on 1 mole of the aryl bromide of the formula III.

Based on 1 molar equivalent of the aryl bromide of the formula III, the base is employed in an amount of from 1 to 5 molar equivalents, preferably from 1.5 to 3 molar equivalents.

In the context of the present invention, suitable bases are, for example, alkali metals or alkaline earth metals, their hydrides, amides, alkoxides, silazanes, carbonates and bicarbonates, and also tertiary amines.

In a preferred embodiment of the process according to the invention, the base used is selected from alkali metal and alkaline earth metal alkoxides, particularly preferably from alkali metal alkoxides, such as sodium alkoxides or potassium alkoxides, and very particularly preferably from sodium alkoxides.

Especially suitable bases for the process according to the invention are C₁-C₄-alkoxides, preferably methoxides and ethoxides, such as sodium methoxide or sodium ethoxide.

It has been found to be particularly advantageous for the carbon-containing radical of the alkoxide used as base and the radical R in the compound of the general formula II to have the same meaning. Accordingly, the carbon-containing radical of the alkoxide and the radical R are particularly preferably methyl or ethyl.

In the process according to the invention, the alkoxide used as base can be employed either in the form of a solid or else as a solution in the corresponding alcohol. The solution generally has a proportion by weight of alkoxide of at least 10% by weight and in particular at least 20% by weight. In the case of alkaline earth metal alkoxides, these can be generated in situ from the alkaline earth metal and the alcohol.

The use of alkoxides as base in the process according to the invention offers substantial advantages compared to the use of sodium hydride, in particular on an industrial scale. Owing to its reactivity, sodium hydride is considerably more difficult to handle and moreover substantially more expensive than, for example, sodium ethoxide or sodium methoxide.

In a special embodiment of the process according to the invention, the malonic ester of the general formula II is initially reacted with the base. A copper salt and the aryl bromide of the formula III are than added to the reaction product. In the process according to the invention, the reaction product of malonic ester and base is generally reacted further without being isolated or purified beforehand.

Here, the reaction temperature for the reaction of the malonic ester II with the base is generally room temperature or above, the upper limit being the boiling point of the components present in the reaction mixture. The reaction temperature is especially in a range of from 20 to 200° C. and in particular in the range from 20 to 90° C. The reaction is usually carried out at atmospheric pressure; however, it may also be carried out at reduced pressure.

In a special embodiment of the process according to the invention, alcohol released during the reaction from the alkoxide and/or added with the base is removed from the reaction mixture by distillation. The distillative removal of the alcohol is preferably essentially complete, i.e. at least 90%, preferably at least 95% and particularly preferably at least 98% of the alcohol present in the reaction mixture is removed by distillation.

The distillative removal of the alcohol is preferably carried out at a temperature above the boiling point of the alcohol at the respective pressure used for the distillation and below the boiling point of the malonic ester of the formula II employed, at the respective pressure used for the distillation. Preferably at least some of the alcohol is removed by distillation under reduced pressure, i.e. at a pressure in the range of from 1 to 1000 mbar, preferably from 2 to 800 mbar and particularly preferably at a pressure of from 5 to 500 mbar.

During the distillation, the pressure is preferably reduced continuously or step-wise.

In the process according to the invention, it has furthermore been found to be advantageous to carry out the distillative removal of the alcohol in a reactor having a stirrer passing close to the wall. Stirrers passing close to the wall are, for example, anchor stirrers or pitched-anchor stirrers. These may additionally be provided with a device for more efficient removal from the wall, such as, for example, wiper blades. Coaxial stirrer systems having two independently operating stirrers, one of the stirrers preferably passing close to the wall, can also be used advantageously.

In a preferred embodiment, the copper salt required for the reaction and the aryl bromide are added to the reaction vessel after the reaction of the malonic ester of the formula II with the base has ended. The addition of the copper salt and the aryl bromide is preferably carried out after the reaction of the malonic ester of the formula II with the base has ended, and in particular after the removal of the alcohol.

In a preferred embodiment of the process according to the invention, the distillative removal of the alcohol is carried out prior to the addition of the copper salt used as catalyst for the substitution reaction at the aryl bromide of the formula III.

In the process according to the invention, the copper salt used as catalyst can be added either in one portion or else a little at a time. In a special embodiment of the process according to the invention, part of the catalyst used is initially charged prior to the addition of the aryl bromide of the formula III, and the remainder of the catalyst is added in aliquots during the course of the reaction.

The copper salt used as catalyst for the substitution reaction at the aryl bromide of the formula III, i.e. for the reaction of the deprotonated malonic ester of the formula II with the aryl bromide of the formula III, preferably has an oxidation state of 1.

Suitable catalysts for the substitution reaction are copper salts of the formula CuX, where X is a monovalent anion, especially Cl, Br, I, or CN. The catalyst used is preferably CuBr or CuCl and particularly preferably CuBr.

In the process according to the invention, the copper salt can be employed either in free form or else in the form of a complex, especially as dialkyl sulfide complex.

The copper salt is usually employed in an amount of from 0.05 to 0.5 molar equivalents, preferably 0.1 to 0.35 molar equivalents, based on one molar equivalent of aryl bromide of the formula III.

The substitution reaction at the aryl bromide of the formula III is preferably carried out in a temperature range of from 40 to 200° C. The upper limit for the reaction temperature is defined by the boiling points of the malonic ester of the formula II used and the aryl bromide of the formula III. Particularly preferably, the substitution reaction is carried out at a temperature of from 60 to 120° C.

In a special embodiment of the process according to the invention, the reaction temperature is increased continuously or step-wise over the course of the substitution reaction.

The substitution reaction at the aryl bromide of the formula III is usually carried out at atmospheric pressure. However, in a special embodiment of the process according to the invention, the substitution reaction may also be carried out under elevated or reduced pressure.

If the substitution reaction is carried out under reduced pressure, it may be possible to remove low-boiling byproducts from the reaction mixture.

In a special embodiment of the process according to the invention, the substitution reaction is carried out with stripping, i.e. passing through an inert gas, such as, for example, nitrogen.

After the reaction has ended, the reaction mixture is preferably subjected to aqueous, particularly preferably aqueous acidic, work-up, i.e. water is added to the reaction mixture or the reaction mixture is added to water, the pH is adjusted, if required, and the aqueous phase obtained is separated from the organic phase which contains the 2-arylmalonic ester of the formula I. The substituted 2-arylmalonic ester of the general formula I is isolated by customary methods such as, for example, crystallization, filtration, extraction and distillation. In a special embodiment of the process according to the invention, an aqueous solution is added to the reaction mixture obtained in the substitution reaction, and the 2-arylmalonic ester is obtained from the resulting organic phase by distillation, preferably under reduced pressure, if appropriate after drying.

In a further embodiment of the process according to the invention, the aryl bromide of the general formula III is additionally provided by bromination of a compound of the general formula Ar-H in which Ar has one of the meanings given above.

The bromination of aryl compounds of the formula Ar-H is known in principle. Usually, the aryl compound of the formula Ar-H or a solution of this compound in an inert solvent is reacted with Br₂ in the presence of a catalyst, especially FeCl₃ or AlCl₃. The Br₂ is preferably employed in substoichiometric amounts, based on the aryl compound to be brominated.

The bromination is usually carried out at a temperature in the range of from −10 to 60° C. The upper limit of the temperature range is defined by the boiling point of Br₂. The reaction is especially carried out at a temperature in the range of from 30 to 50° C.

In a special embodiment of the process according to the invention, the bromination is carried out neat, i.e. without addition of an inert solvent.

After the bromination has ended, the reaction mixture is preferably subjected to aqueous work-up, particularly preferably in the presence of sodium bisulfite. The aryl bromide of the general formula III is isolated by customary methods such as, for example, extraction and distillation.

Advantageously, a compound of the formula Ar-H which may have been formed during the reaction of the aryl bromide of the general formula III and any unreacted aryl bromide of the general formula III may be removed and brominated again or fed into the work-up of the bromination.

In an advantageous manner, the process according to the invention is also suitable for being carried out in the form of a continuous process. Accordingly, the present invention furthermore provides a process according to the invention in which at least some of the reactions or work-ups are carried out continuously. In a special embodiment of the process according to the invention, the entire process is carried out continuously.

In the context of the present invention, the term “continuous process” refers to a process in which at least one of the compounds involved in the reaction is fed continuously to the reaction and at least one of the intermediates or products of the reaction is removed continuously in the form of a discharge from a reaction mixture. Starting materials and intermediates obtained by separating reaction mixtures removed as discharge may advantageously be recycled to the process steps in question. Suitable reactors for continuous reaction are known to the person skilled in the art and described, for example, in Ullmanns Enzyklopädie der technischen Chemie [Ullmanns Encyclopedia of Industrial Chemistry], Vol. 1, 3rd ed., 1951, p. 743 ff.

In the compounds of the general formula I which can be prepared by the process according to the invention, the radical Ar is preferably selected from the group consisting of phenyl, pyridin-2-yl, pyridin-4-yl, pyrazin-2-yl, pyrimidin-2-yl, pyrimidin-4-yl, pyridazin-3-yl and pyridazin-4-yl, where each of the carbons contained in the radicals mentioned above may optionally carry a substituent R^{A}. Ar is particularly preferably selected from the group consisting of optionally substituted phenyl, pyridin-2-yl and pyridin-4-yl. Ar is especially optionally substituted phenyl.

Furthermore, substituents R^{A} optionally present in the compounds of the general formula I are independently of one another preferably selected from the group consisting of fluorine, chlorine, cyano, C₁-C₄-alkyl, C₁-C₄-haloalkyl, C₁-C₄-alkoxy and C₁-C₄-haloalkoxy. Particularly preferably, R^{A} is fluorine or chlorine.

Preference is also given to compounds of the general formula I in which two adjacent substituents R^{A} together with the carbon atoms to which they are attached form a phenyl ring.

In a particularly preferred embodiment of the present invention, the process according to the invention is used for preparing 2-arylmalonic esters of the general formula I in which Ar is phenyl which optionally comprises 1, 2 or 3 substituents R^{A} independently of one another selected from the group consisting of fluorine and chlorine.

Below, the present invention is illustrated by non-limiting examples.

### WORKING EXAMPLES

### Example B.1

### Preparation of 2,4,6-trifluorobromobenzene

1,3,5-Trifluorobenzene (400.1 kg, 3029 mol) is initially charged in a 1 m³ reactor, anhydrous iron(III) chloride (FeCl₃, 3.78 kg, 23.3 mol) is added and the mixture is warmed to 40° C. Bromine (372.6 kg, 2330 mol) is then added over a period of 32 h. After the addition has ended, the reaction solution is stirred at 40° C. for 2 h. The reaction solution is then cooled to 15° C. and transferred into a stirring vessel with water (200 kg). The aqueous phase is removed and the organic phase is washed with water (200 kg). By addition of sodium hydroxide solution (7.0 kg, 25% strength aqueous solution), the pH is adjusted to 8. At the same time, sodium bisulfite (7.0 kg, 38% strength aqueous solution) is added. The phases are separated and the organic phase is then rectified under reduced pressure. This gave 2,4,6-trifluorobromobenzene in a yield of 97.5% (479.3 kg, 2272 mol), based on the bromine employed.

During the rectification, unreacted 1,3,5-trifluorobenzene is recovered and may, if desired, be recycled to the bromination.

### Example B.2a

### Preparation of diethyl 2,4,6-trifluorophenylmalonate

### (Base: NaOEt; Catalyst: 0.23 Equivalents of CuBr)

Dry diethyl malonate (2883.1 g, 18.00 mol) is initially charged at room temperature in a 6 l apparatus with anchor stirrer, and solid sodium methoxide (673.7 g, 9.90 mol) is added. Owing to the heat of reaction released, the internal temperature rises to about 60° C. After the reaction has ended, most of the ethanol formed is distilled off under reduced pressure (400 mbar) with simultaneous increase of the temperature from 60 to 80° C. At 80° C., the pressure is reduced step-wise to 10 mbar. At atmospheric pressure, the residue is then cooled to 75° C., and CuBr (148.5 g, 1.04 mol) and 2,4,6-trifluoro-bromobenzene (949.4 g, 4.5 mol) are added successively over a period of 20 min. After a further 8 h at 75° C., the temperature is kept at 85° C. for 2 h and finally at 100° C. for a further 2 h. After the reaction has ended, the reaction mixture is cooled to 15° C. and added with stirring to a mixture, cooled to 10° C., of hydrochloric acid (36% strength, 732.2 g) and water (1451.0 g). The reaction mixture obtained is filtered. The phases of the filtrate are separated, water (1455 g) is then added to the organic phase and the pH is adjusted to 3.5-4 by addition of potassium carbonate (28.7 g, 50% strength solution in water). The phases are again separated, and the organic phase is then rectified under reduced pressure (0.5 mbar). Diethyl 2,4,6-trifluorophenylmalonate was obtained in a yield of 81.0% (1057.9 g, 3.645 mol) (b.p. 83° C. at 0.5 mbar, m.p. 52° C.).

The results summarized in table 1 were obtained for an analogous procedure by varying the molar ratio of diethyl malonate (DEM) to sodium ethoxide (NaOEt).

TABLE 1

| | DEM/ | NaOEt/ | | | Yield after | |
|---|---|---|---|---|---|---|
| | NaOEt | BrF₃Ph*** | | | aqueous | Yield after |
| Example | [mol/mol] | [mol/mol] | Conversion | Selectivity | work-up | distillation |
| B.2a | 1.8 | 2.2 | 99.5% | 83.5% | 83.1% | 81.0% |
| B.2b₁ | 2.0 | 2.2 | 99.2% | 83.2% | 82.5% | 80.1% |
| B.2b₂* | 2.0 | 2.2 | 95.9% | 81.9% | 78.5% | 75.6% |
| B.2c | 2.3 | 2.2 | 98.6% | 80.5% | 79.4% | 76.8% |
| B.2d | 2.7 | 2.2 | 97.2% | 79.3% | 77.1% | 74.8% |
| B.2e₁ | 3.2 | 2.2 | 94.6% | 78.2% | 74.0% | 71.4% |
| B.2e₂* | 3.2 | 2.2 | 90.8% | 77.7% | 70.6% | 67.8% |
| B.2e₃** | 3.2 | 2.2 | 81.4% | 76.9% | 62.6% | 60.2% |
| B.2f | 3.6 | 2.2 | 92.2% | 76.7% | 70.7% | 67.8% |
| B.2g | 6.8 | 2.2 | 85.7% | 72.8% | 62.4% | 61.0% |

*only 90% of the ethanol was distilled off
**the ethanol was not distilled off
***BrF₃Ph = 2,4,6-trifluorobromobenzene

### Example B.3a

### Preparation of diethyl 2,4,6-trifluorophenylmalonate

### (Base: NaOEt, 21% Strength Solution in Ethanol)

Dry diethyl malonate (2883.1 g, 18.00 mol) is initially charged at room temperature in a 6 l apparatus with anchor stirrer, and NaOEt (3208.1 g, 9.90 mol) is added as a 21% strength solution in ethanol. The pressure is then reduced to 300 mbar, and ethanol is removed by distillation with simultaneous increase of the temperature from room temperature to 80° C. At a temperature of 80° C., the pressure is reduced step-wise to 10 mbar. After cooling of the residue to 75° C., CuBr (148.5 g, 1.04 mol) and 2,4,6-tri-fluorobromobenzene (949.4 g, 4.5 mol) are added successively over a period of 20 min. After a further 8 h at 75° C., the temperature is kept at 85° C. for 2 h and finally at 100° C. for a further 2 h. After the reaction has ended, the reaction solution is cooled to 15° C. and added with stirring to a mixture, cooled to 10° C., of hydrochloric acid (36% strength, 732.2 g) and water (1451.0 g). The reaction mixture is filtered. The phases of the filtrate are separated, water (1454 g) is then added to the organic phase and the pH is adjusted to 3.5-4 by addition of potassium carbonate (30.4 g, 50% strength solution in water). The phases are again separated, and the organic phase is then rectified under reduced pressure (0.5 mbar). Diethyl 2,4,6-trifluorophenylmalonate was obtained in a yield of 80.7% (1054.2 g, 3.632 mol) (b.p. 83° C. at 0.5 mbar, m.p. 52° C.).

The results summarized in table 2 were obtained for an analogous procedure by varying the molar ratio of diethyl malonate (DEM) to sodium ethoxide (NaOEt) and the amount of catalyst.

TABLE 2

| | Ratio DEM/ | Ratio NaOEt/ | Ratio CuBr/ | | | Yield after | |
|---|---|---|---|---|---|---|---|
| | NaOEt | BrF₃Ph | BrF₃Ph | | | aqueous | Yield after |
| Example | [mol/mol] | [mol/mol] | [mol/mol] | Conversion | Selectivity | work-up | distillation |
| B.3a | 1.82 | 2.2 | 0.23 | 99.7% | 83.5% | 83.2% | 80.7% |
| B.3b | 2.00 | 2.2 | 0.23 | 99.4% | 83.0% | 82.5% | 79.9% |
| B.3c | 3.18 | 2.2 | 0.23 | 95.6% | 78.4% | 75.0% | 72.3% |
| B.3d | 6.82 | 2.2 | 0.23 | 86.7% | 71.9% | 62.3% | 60.3% |
| B.3f | 2.00 | 2.2 | 0.14 | 97.5% | 83.7% | 81.6% | 79.3% |
| B.3g | 2.00 | 2.2 | 0.35 | 99.8% | 79.4% | 79.2% | 76.6% |
| B.3h*⁾ | 2.00 | 2.2 | 0.23 | 98.3% | 75.8% | 74.5% | 72.2% |
| B.3i**⁾ | 2.00 | 2.2 | 0.23 | 87.4% | 84.1% | 73.5% | 70.7% |

*⁾CuBr was used in the form of a CuBr/dimethyl sulfide complex
**⁾CuCl was used instead of CuBr

### Comparative Example VB.4

### Preparation of diethyl 2,4,6-trifluorophenylmalonate (According to DE 19938736)

Dry diethyl malonate (1212.3 g, 7.57 mol) is initially charged in dry dioxane (3 l) at 50° C. Sodium ethoxide (441.0 g, 6.48 mol) is added a little at a time over a period of 1 h. After a further hour at 50-55° C., the mixture is distilled until a head temperature which corresponds to the boiling point of pure dioxane is reached. The residue is cooled to 90° C., and copper(I) bromide (176 g, 1.23 mol), copper(I) iodide (176 g, 0.924 mol) and 2,4,6-trifluorobromobenzene (1238.5 g, 5.87 mol) are added. After a further 15 hours under reflux conditions, the reaction mixture is cooled to 15° C. and a mixture, cooled to 10° C., of water (1465 ml) and concentrated hydrochloric acid (36% strength, 1172 ml) is added. The reaction mixture is then filtered and diluted with water (2.5 l), and the filtrate is extracted with tert-butyl methyl ether (2 times with in each case 1.5 l). The organic phase is washed twice with water (1.5 l), dried and distilled under reduced pressure (0.5 mbar). Diethyl 2,4,6-trifluorophenylmalonate was obtained in a yield of 42.4% (722.3 g, 2.49 mol) (b.p. 83° C. at 0.5 mbar).

### Example B.5a

### Preparation of dimethyl 2,4,6-trifluorophenylmalonate

### (Base: Sodium Methoxide (NaOMe), 30% Strength Solution In Methanol; Catalyst: 0.23 Equivalents of CuBr)

At room temperature, dry dimethyl malonate (3630.7 g, 27.48 mol) is initially charged in a 6 l apparatus with anchor stirrer, and sodium methoxide (1484.5 g, 8.24 mol, 30% strength solution in methanol) is added. At reduced pressure (500 mbar) and with simultaneous temperature increase from 35 to 80° C., methanol is then distilled off. At 80° C., the pressure is reduced step-wise to 10 mbar. The residue is cooled to 75° C., and CuBr (113.4 g, 0.789 mol) and 2,4,6-trifluorobromobenzene (724.7 g, 3.435 mol) are added successively over a period of 20 min. After a further 8 h, at 75° C., the temperature is kept at 85° C. for 2 h and finally at 100° C. for 2 h. After the reaction has ended, the reaction mixture is cooled to 15° C. and added with stirring to a mixture, cooled to 10° C., of hydrochloric acid (36% strength, 610.2 g) and water (1209.2 g). The reaction mixture is filtered. The phases of the filtrate are separated, water (1210.0 g) is then added to the organic phase and the pH is adjusted to 3.5-4 by addition of potassium carbonate (50% strength aqueous solution, 31.9 g). The phases are separated again and the dimethyl 2,4,6-trifluorophenylmalonate content of the organic phase is then determined by quantitative HPLC analysis. This gave 3930.8 g of organic phase having a dimethyl 2,4,6-trifluorophenylmalonate content of 18.9% by weight. This corresponds to a dimethyl 2,4,6-trifluorophenylmalonate yield of 82.5% (742.9 g, 2.834 mol).

The results summarized in table 3 were obtained for an analogous procedure by varying the molar ratio of dimethyl malonate (DMM) to sodium methoxide (NaOMe).

TABLE 3

| | DMM/ | NaOMe/ | | | Yield after |
|---|---|---|---|---|---|
| | NaOMe | BrF₃Ph | | | aqueous |
| Example | [mol/mol] | [mol/mol] | Conversion | Selectivity | work-up |
| B.5a | 3.33 | 2.4 | 98.4% | 83.9% | 82.5% |
| B.5b₁ | 4.17 | 2.4 | 95.0% | 79.4% | 75.4% |

### Example B.6

### Preparation of diethyl 2,4-dichlorophenylmalonate (base: sodium ethoxide (NaOEt); catalyst: 0.23 equivalents of CuBr)

At room temperature, dry diethyl malonate (1139.7 g, 7.12 mol) is initially charged in a 1.6 l apparatus with anchor stirrer, and sodium ethoxide (244.1 g, 3.59 mol) is added as a solid. Owing to the energy of reaction released, the internal temperature increases to about 60° C. After the reaction is ended, the ethanol formed is distilled off under reduced pressure (400 mbar) and simultaneous increase of the temperature from 60 to 80° C. Then, at 80° C., the pressure is gradually reduced to 10 mbar. At atmospheric pressure, the residue is cooled to 75° C., and CuBr (53.3 g, 0.37 mol) and 2,4-dichlorobromobenzene (361.2 g, 1.60 mol) are added successively over a period of 20 minutes. After a further 12 hours at 75° C. and 2 hours at 90° C., the reaction mixture is cooled to 15° C. and, with stirring, added to a mixture, cooled to 10° C., of hydrochloric acid (36% strength, 260.9 g) and water (512.8 g). The reaction mixture obtained is filtered. Following separation of the phases of the filtrate, water (514.0 g) is added to the organic phase, and the pH is adjusted to 4 by addition of potassium carbonate (4.0 g, 50% strength solution in water). The phases are separated again, and the organic phase is then freed from volatile components under reduced pressure (0.5 mbar) and up to an internal temperature of 123° C. According to quantitative ¹H-NMR spectroscopy, 83.7% of residue (501.5 g) consisted of diethyl 2,4-dichlorophenylmalonate. This corresponds to a diethyl 2,4-dichlorophenylmalonate yield of 86.0%.

### Example B.7

### Preparation of diethyl 3,4,5-trifluorophenylmalonate (base: sodium ethoxide (NaOEt); catalyst: 0.23 equivalents of CuBr)

At room temperature, dry diethyl malonate (1140.2 g, 7.12 mol) is initially charged in a 1.6 l apparatus with anchor stirrer, and sodium ethoxide (244.5 g, 3.59 mol) is added as a solid. Owing to the energy of reaction released, the internal temperature increases to about 60° C. After the reaction has ended, the ethanol formed is distilled off under reduced pressure (400 mbar) and simultaneous increase of the temperature from 60 to 80° C. At 80° C., the pressure is gradually reduced to 10 mbar. At atmospheric pressure, the residue is then cooled to 75° C., and CuBr (53.4 g, 0.37 mol) and 3,4,5-trifluorobromobenzene (338.4 g, 1.60 mol) are then added successively over a period of 20 minutes, and the mixture is kept at 75° C. for another 18 hours. After the reaction has ended, the reaction mixture is cooled to 15° C. and, with stirring, added to a mixture, cooled to 10° C., of hydrochloric acid (36% strength, 260.9 g) and water (512.8 g). The reaction mixture obtained is filtered. Following separation of the phases of the filtrate, water (512.8 g) is added to the organic phase, and the pH is adjusted to 3.8 by addition of potassium carbonate (5.9 g, 50% strength solution in water). The phases are separated again, and the organic phase is then freed from volatile components under reduced pressure (0.5 mbar) up to an internal temperature of 127° C. According to quantitative ¹⁹F-NMR spectroscopy, 79.6% of the residue (478.4 g) consisted of diethyl 3,4,5-trifluorophenylmalonate. This corresponds to a diethyl 3,4,5-trifluorophenyl-malonate yield of 82.0%.

1-11. (canceled)

12. A process for preparing a compound of formula I 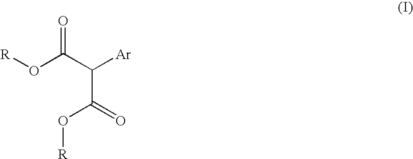
in which
R is C₁-C₆-alkyl or C₁-C₄-alkoxy-C₁-C₄-alkyl; and
Ar is phenyl or a heteroaromatic 5- or 6-membered ring comprising, as ring members, 1 or 2 heteroatoms selected from the group consisting of N, S and O, where each carbon atom present in the radicals mentioned above optionally carries a substituent R^{A}, where
R^{A} independently of one another is fluorine, chlorine, cyano, nitro, C₁-C₄-alkyl, C₁-C₄-haloalkyl, C₁-C₄-alkoxy, C₁-C₄-haloalkoxy, C₁-C₆-alkoxycarbonyl, C₁-C₆-alkylaminocarbonyl or di-(C₁-C₆-alkyl)aminocarbonyl; or
two adjacent substituents R^{A} together with the carbon atoms to which they are attached form an aromatic or partially saturated optionally substituted 5- to 7-membered ring; and
comprising reacting a malonic ester of the general formula II 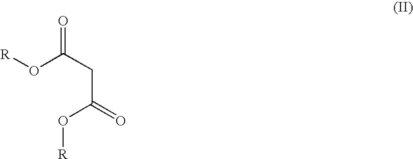
with a base and an aryl bromide of the formula III
Ar-Br (III)
in the presence of a copper salt, wherein from 0.1 to 0.65 molar equivalents of the base per molar equivalent of the malonic ester of the formula II is employed to obtain the compound of formula I.

13. The process of claim 12 where the reaction is carried out essentially without addition of an inert solvent.

14. The process of claim 12 where the base is selected from the group consisting of alkali metal alkoxides and alkaline earth metal alkoxides.

15. The process of claim 14 where the alkoxide of said base is the same alkoxide group present in the compound of formula II when R is C₁-C₆-alkyl.

16. The process of claim 15 wherein the alkoxide is methoxy or ethoxy.

17. The process of claim 16 wherein an alcohol released during the reaction is removed by distillation from the reaction mixture.

18. The process of claim 12 where the malonic ester of the general formula II is reacted with the base and the resulting reaction product is reacted in the presence of the copper salt with the aryl bromide of the general formula III.

19. The process of claim 18 where the removal of the alcohol is carried out prior to the addition of the copper salt.

20. The process of claim 12 where additionally the aryl bromide of the general formula III is obtained from bromination of a compound of the general formula Ar-H.

21. The process of claim 20 where a compound of the formula Ar-H which may have been formed during the reaction of the aryl bromide of the general formula III and any unreacted aryl bromide of the general formula III are separated off and recycled in the bromination step.

22. The process of claim 12 in which Ar is phenyl which optionally comprises 1, 2 or 3 substituents R^{A} independently of one another selected from the group consisting of fluorine and chlorine.
